(19) United States
(12) Patent Application Publication
(10) Pub. No.: US 2021/0330359 A1
(43) Pub. Date: Oct. 28, 2021

(54) BURR HOLE COVER

(71) Applicant: OsteoMed LLC, Addison, TX (US)

(72) Inventors: Benjamin Casey, Richardson, TX (US); Devid R. Zille, Addison, TX (US); Justin L. Rowland, Atlanta, GA (US)

(21) Appl. No.: 17/240,498

(22) Filed: Apr. 26, 2021

Related U.S. Application Data
(60) Provisional application No. 63/016,113, filed on Apr. 27, 2020.

(51) Int. Cl. A61B 17/68; B33Y 80/00

(52) U.S. Cl. CPC A61B 17/686; A61B 2017/00526; B33Y 80/00

(57) ABSTRACT

A burr hole cover is provided that is constructed to be positioned within a recessed region formed on a patient's skull in order to conceal a burr hole. The burr hole cover may be affixed to the patient's skull in the recessed region. For instance, surgical fastening devices may be inserted through apertures defined in the burr hole cover. The burr hole cover may have a thickness and curvature that enables it to be substantially flush with the surface of the skull when affixed in the recessed region. The burr hole cover's curvature follows the contour of the patient's skull. The burr hole cover may include one or more perforations sized to allow air (or moisture) to flow towards the burr hole, which promotes skin/bone growth. Some of the perforations may be large enough to permit access to the brain with leads and/or drainage devices.

### PRIORITY CLAIM

The present application claims priority to and the benefit of U.S. Provisional Application 63/016,113, filed Apr. 27, 2020, the entirety of which is herein incorporated by reference.

### BACKGROUND

Craniotomy is a surgical procedure generally performed to treat neurosurgical conditions and diseases. A craniotomy may involve forming one or more burr holes—which are small holes (e.g., 8-10 mm in diameter) created in the skull through to the level of the dura—to provide a surgeon access to the brain. The access allows a surgeon to perform a desired neurosurgical procedure (e.g., deep brain stimulation). However, forming burr holes often leads to skull defects resulting in scalp depressions that are usually unacceptable to a patient from a cosmetic perspective. In some instances, the scalp depressions may aggravate over time with the wound swelling in the early stages and the soft tissues weakening in the later stages. To prevent scalp depressions and other complexities caused by them, surgeons often conceal burr holes using burr hole covers.

Numerous different designs of burr hole covers made from different materials have been employed. However, each one of them has issues. For instance, burr hole covers made of autologous bone, muscle, or fat tissue, although highly biocompatible, are linked with donor site complications, are time consuming, and are difficult to apply on burr holes. On the other hand, polymethyl methacrylate (PMMA)-based covers can be applied to burr holes, but securing them to burr holes is time consuming. PMMA-based covers may also have a thermal reaction which may be highly toxic for surrounding tissues. In contrast, mineral grafts such as hydroxyapatites (HAs) are not toxic to the surrounding tissues and have osteo-conductive properties, but they are often too brittle and reabsorb in the presence of cerebrospinal fluid (or any liquid).

Titanium-based burr hole covers have shown promise; however, they are undesirably more pliable than necessary and lack required rigidity. Further, typical titanium-based burr hole covers generally require one or more surgical screws to secure the cover with the underlying skull bone. However, employing surgical screws brings about cosmetic challenges of their own. For example, the head of the surgical screws may protrude out from the scalp and appear as undesirable crests, thus creating cosmetic complexities for a patient. In addition to the cosmetic challenges, employing surgical screws may bring about other complexities. For example, the screw head may erode the underlying skull bone, which may displace the burr hole cover from its desired position.

### SUMMARY

The present disclosure generally relates to burr hole covers and methods for affixing such burr hole covers to a patient's skull. More specifically, a burr hole cover is provided that is configured to conceal a burr hole where the cover is positioned within a recessed region formed on a patient's skull. The provided burr hole cover may have an outer surface that follows the contour of the patient's skull. When affixed to the patient's skull, the burr hole cover may be substantially flush with the surface of the skull. In some aspects, the burr hole cover may include one or more apertures configured to receive surgical fastening devices (e.g., surgical screws, wires, etc.) in order to allow surgeons to affix the burr hole cover in the recessed region. In some aspects, the burr hole cover may include one or more perforations large enough (e.g., 2-5 mm in diameter) to permit access to the brain with a medical instrument. To illustrate, the burr hole cover may include perforations designed to allow surgeons to insert a medical instrument, such as leads (e.g., deep brain stimulation lead) and/or drainage devices into the brain. In some aspects, the burr hole cover may include one or more relatively small perforations (e.g., 0.5-1 mm in diameter) designed to allow air (or moisture) to flow towards the burr hole, which promotes skin/bone growth.

In some aspects, the burr hole cover may be patient specific in that the burr hole cover may be individually custom designed for every patient according to the contour of the patient's skull. The custom design of the burr hole cover may be created using various medical imaging techniques (e.g., CT scans, MRI scans, and the like). In other aspects, the burr hole cover may not be patient specific. In such other aspects, burr hole covers may be constructed based on age, gender, or other generic physical makeup of the human anatomical structure. The present disclosure also describes various aspects of methods for manufacturing the provided burr hole covers. For instance, burr hole covers may be manufactured using 3D printing techniques, injection molding techniques, and the like.

The foregoing has outlined rather broadly the features and technical advantages of the aspects so that the detailed description of the aspects that follows may be better understood. Additional features and advantages of the aspects disclosed in this application will be described hereinafter which form the subject of the claims of the application. It should be appreciated by those skilled in the art that the conception and specific aspect disclosed may be readily utilized as a basis for modifying or designing other structures for carrying out the same purposes of the present application. It should also be realized by those skilled in the art that such equivalent constructions do not depart from the spirit and scope of the aspects in this application as set forth in the appended claims. The novel features which are believed to be characteristic of the aspects, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 illustrates a perspective view of an example burr hole positioned within a recessed region, in accordance with aspects of the present disclosure.

FIG. 2A illustrates a perspective view of a burr hole cover, in accordance with aspects of the present disclosure.

FIG. 2B illustrates a side view of the burr hole cover of FIG. 2A, in accordance with aspects of the present disclosure

FIG. 3 illustrates a perspective view of the burr hole cover of FIG. 2A affixed in the recessed region of FIG. 1, in accordance with aspects of the present disclosure.

FIG. 4 illustrates a flowchart of a method for affixation of a burr hole cover within a recessed region formed in a skull bone, in accordance with aspects of the present disclosure.

### DETAILED DESCRIPTION

The present disclosure describes a burr hole cover and a method for affixing the burr hole cover to a patient's skull to cover a burr hole formed in the patient's skull. The provided burr hole cover may be affixed within a recessed region formed in the patient's skull. In some aspects, the recessed region may be formed on the patient's skull before the burr hole is drilled within the recessed region. In other aspects, the burr hole may be drilled before the recessed region is formed around the burr hole. In yet another aspect, both the recessed region and the burr hole may be formed simultaneously using specific bits configured to form both the recessed region and the burr hole. In any of these aspects, the recessed portion is positioned around the burr hole. Stated differently, the burr hole is positioned within the recessed region. When the provided burr hole cover is affixed within the recessed region, the provided burr hole cover conceals the burr hole and is flush with the surface of the skull. Further, surgical fixation devices used to affix the provided burr hole cover are flush, or recessed within, the burr hole cover's top surface when the provided burr hole cover is affixed within the recessed region. Accordingly, the presently disclosed burr hole cover provides for cosmetic improvements to typical devices which are raised, and/or have extensions and utilize screws on the top surface of the skull.

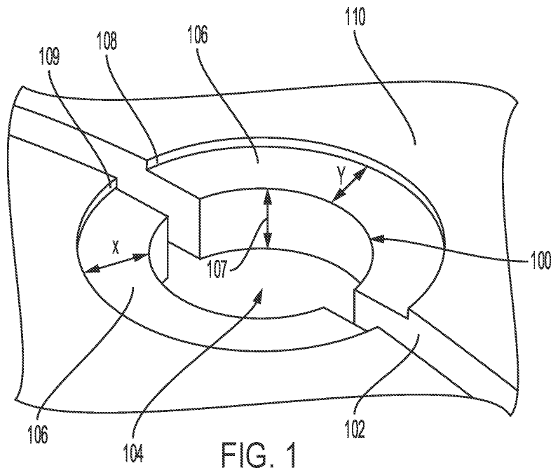

FIG. 1 illustrates a recessed region 106 positioned around a burr hole 100. The recessed region 106 may be positioned around the burr hole 100 such that the recessed region 106 extends away from the mouth or center of the burr hole 100. The burr hole 100 extends to the level of the dura, exposes the brain, and provides the surgeon access to the brain via the region 104. In some instances, a cranial drill, also known as a craniotome, may be used for creating the burr hole 100. The drill itself can be manually or electrically driven, and may include a hand piece, a drill and/or driver bit. The craniotome may be configured to provide torque to the drill bit which further creates the burr hole 100 and the region 104. To illustrate, when a drill bit is pressed against the skull bone and rotated at rates, for instance, from hundreds to thousands of revolutions per minute, the drill bit may cut/chip off skull bone material and create the burr hole.

It is noted that FIG. 1 shows the recessed region 106 having a circular shape and following the circumference/boundary of the burr hole 100. However, the shape of the recessed region 106 is not limited to a circular shape, and the recessed region 106 can have any suitable shape as long as the region extends away from the mouth of the burr hole 100 and is positioned around the burr hole 100. For example, the recessed region 106 may be positioned around the burr hole 100 in the shape of a square or another suitable shape. It is appreciated that circular shapes are preferred due to the ability to utilize a drill to form the recessed region 106, but other tools and methods to form a recessed region may be utilized in accordance with embodiments.

The example recessed region 106 shown in FIG. 1 defines a break at a channel region 102. The channel 102 is a tract of a hollow skull bone connecting the burr hole 100 with other burr holes that may be present on the patient's skull. In procedures without the presence of the channel region 102, the recessed region 106 may not define a break and may rather be continuous.

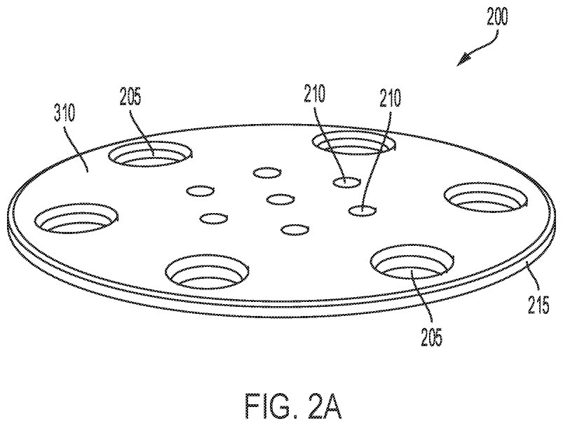

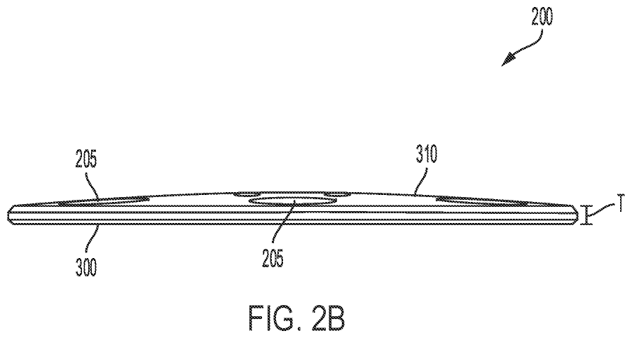

The recessed region 106 may have a depth 108 and a depth 109. The depth 108 and the depth 109 are distances of a vector normal to the skull bone 110 and the base surface of the recessed region 106. In some aspects, the depth 108 and/or the depth 109 of the recessed region 106 may be about 0.5-1 mm. The portion of the recessed region 106 defining the depth 108 and the depth 109 may be referred to as a wall of the recessed region 106. In some aspects, the depths 108 and 109 of the recessed region 106 may be defined prior to surgery. In at least some aspects, the depths 108 and 109 may depend on the thickness of the burr hole cover (e.g., the burr hole cover 200 of FIG. 2A) that is to be installed within the recessed region 106. For instance, the thickness of the burr hole cover to be installed in the recessed region 106 may be uniform, and in such instances, the depths 108 and 109 may be uniform throughout the recessed region 106. In other instances, the thickness of the burr hole cover to be installed in the recessed region 106 may be non-uniform, and in such instances, the depths 108 and 109 may be different (or non-uniform). Such differences in thickness may be utilized to provide varying levels of structural integrity/flexibility, to account for different anatomical regions of placement, and/or provide for cosmetic benefits.

In various aspects, the depths 108 and 109 may depend on a thickness 107 of the skull bone 110 underlying the recessed region 106. The thickness 107 of the skull bone 110 may depend on the location. For instance, the frontal portion of the skull bone 110 may have a thickness 107 of about 6.3 mm; the temporal portion of the skull bone 110 may have a thickness 107 of about 3.9 mm; the occipital portion of the skull bone 110 may have a thickness 107 of about 7.75-9.5 mm; the parietal portion of the skull bone 110 may have a thickness 107 of about 5.8 mm; and the central frontal portion of the skull bone 110 may have a thickness 107 of about 8.1 mm. As such, the thicker regions may tolerate a recessed region 106 of relatively larger depth 108 and 109, whereas the thinner regions may tolerate a recessed region 106 of relatively smaller depth 108 and 109. As such, the depth 108 and 109 of the recessed region 106 may also vary based on the location at which the burr hole 100 is drilled. The recessed region 106 may receive surgical fastening devices to affix the burr hole cover within the recessed region 106. As such, in various aspects, the depths 108 and 109 may also depend on the dimensions (e.g., size of the screw head) of the surgical fastening devices used to affix the burr hole cover within the recessed region 106.

In various aspects, the base surface of the recessed region 106 includes a width X and a width Y. The width X and the width Y are each a distance between a lip of the burr hole 100 and the wall of the recessed region 106. The width X and/or the width Y of the recessed region 106, in various aspects, may depend on the diameter of the burr hole 100. In some aspects, the width X and/or the width Y may depend on the dimensions (e.g., diameter of the screw head) of the surgical fastening devices used to install a burr hole cover within the recessed region 106. In some aspects, the width X and/or the width Y may be uniform. In other aspects, the width X and/or the width Y may be non-uniform.

FIGS. 2A and 2B illustrate a perspective and side view, respectively, of an example burr hole cover 200. The burr hole cover 200 includes a top surface 310 opposite a contact surface 300. When the burr hole cover 200 is installed within the recessed region 106, the contact surface 300 contacts the recessed region 106. The burr hole cover 200 includes an outer perimeter or edge 215. The edge 215 of the burr hole cover 200 may contact a wall of the recessed region 106 when the burr hole cover 200 is installed within the recessed region 106. The burr hole cover 200 may include a thickness T between the top surface 310 and the contact surface 300. In some aspects, the burr hole cover 200 may have a constant thickness T throughout. For instance, as shown in FIG. 2B, the top surface 310 may be curved. In such instances, the contact surface 300 may also be curved to maintain a constant thickness T throughout the burr hole cover 200. In other aspects, the burr hole cover 200 may have areas with a greater thickness T than other areas (e.g., a non-uniform thickness T). In such aspects, differences in thickness T of the burr hole cover 200 may be utilized to provide varying levels of structural integrity/flexibility, to account for different anatomical regions of placement, and/or provide for cosmetic benefits.

In at least some aspects, the burr hole cover 200 may include one or more apertures 205. Each aperture 205 may extend from the top surface 310 to the contact surface 300. In at least some aspects, one or more of the apertures 205 may be constructed to receive surgical fixation devices (e.g., surgical screws, wires, and the like) that affix the burr hole cover 200 in the recessed region 106. Each aperture 205 may be constructed so as to enable a head of a surgical fixation device (e.g., screw head of a surgical screw) to be flush or contained within the aperture 205. Stated differently, each aperture 205 may include sidewalls that receive a countersunk head of a surgical fastening device such that the countersunk head sinks in the aperture 205. In some aspects, the sidewalls of an aperture 205 may be inwardly beveled. Enabling a fixation device to be flush within the aperture 205 prevents screw head protrusion, and therefore prevents cosmetic complexities caused therefrom. In some aspects, one or more of the apertures 205 may be constructed to receive locking screws (e.g., the apertures 205 may be reverse threaded) to prevent the screws from backing out once installed.

In some aspects, the burr hole cover 200 may include one or more perforations 210. The perforations 210 may be sized (e.g., 0.5-1 mm in diameter) to allow air/moisture to flow into the underlying areas of the burr hole cover 200 so as to prevent skin damage and promote skin and/or bone growth. In some aspects, one or more of the perforations 210 may be sized (e.g., 2-5 mm in diameter) so as to allow surgeons to insert a medical instrument, such as leads (e.g., deep brain stimulation leads) and/or surgical site drainage channels (e.g., drainage pipes), and the like. In one aspect, some of the perforations 210 are sized to allow air/moisture to flow into the underlying areas while some of the perforations 210 are sized to allow surgeons to insert leads, surgical site drainage channels, or the like.

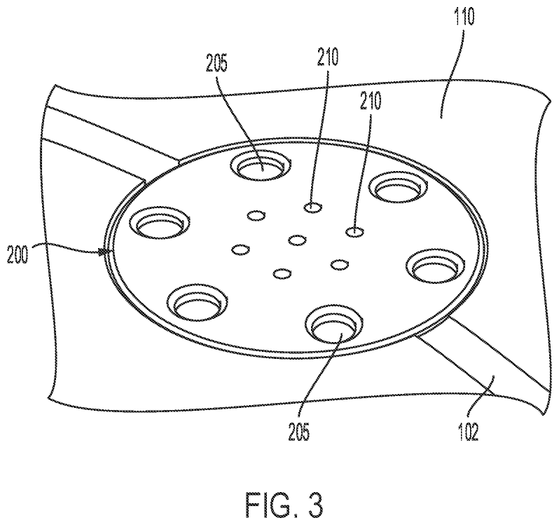

FIG. 3 illustrates the example burr hole cover 200 positioned within the recessed region 106. As noted above, the burr hole cover 200 is constructed to be positioned in the recessed region 106 such that it sits substantially flush with the surface of the skull bone 110 and prevents cosmetic complexities. Stated differently, the burr hole cover 200 has a thickness T (whether constant or non-constant) and curvature such that the top surface 310 is substantially flush with the surface of the skull bone 110 when the burr hole cover 200 is positioned within the recessed region 106. Additionally, as described above, each of the surgical fixation devices used to affix the burr hole cover 200 are flush or recessed within an aperture 205 to help prevent cosmetic complexities. It is noted that the goal is for the burr hole cover 200 to be perfectly flush with the skull bone 110, but it should be appreciated that that the burr hole cover 200 may not always be perfectly flush with the skull bone 110 due to variance based on, e.g., specific patient anatomy, limitations on the precision of the hole, etc. Even when not perfectly flush, a substantially flush burr hole cover 200 improves upon typical devices that are raised, and/or have extensions and utilize screws on the top surface of the skull.

In some aspects, the burr hole cover 200 may be constructed to fit within the recessed region 106 such that the lateral clearance between the edge 215 of the burr hole cover 200 and the wall of the recessed region 106 is negligible or substantially low (e.g., between about 0.00 mm and 0.04 mm). The burr hole cover 200 may have the same shape as the recessed region 106. For example, in embodiments in which the recessed region 106 has a square shape, the burr hole cover 200 has a similar square shape.

In some aspects, the burr hole cover 200 may be patient-specific in that the burr hole cover 200 may be individually constructed for a particular patient according to the patient's skull contour. In such aspects, the burr hole cover 200 may be created with the use of various medical imaging techniques (e.g., CT scans, MM scans, and the like). In such aspects, the burr hole cover 200 may be constructed to have a curvature such that, when placed within the recessed region 106, the burr hole cover 200 follows the curvature of the patient's skull. In some aspects, the burr hole cover 200 may not be patient-specific. For example, the burr hole cover 200 might not be constructed for a specific patient, but is rather constructed based on body types, gender, or other generic characteristics or physical makeup of humans (e.g., age). In such aspects, the same construction of the burr hole cover 200 can be used with different patients.

The burr hole cover 200, in some examples, may be manufactured using 3D printing techniques, injection molding, and the like. An illustrative method for manufacture of the burr hole cover 200 is now described. In one aspect, the burr hole cover 200 may be manufactured using additive technology or freeform fabrication. In some examples, the burr hole cover 200 may be 3D printed of metal (e.g., titanium), metal alloys (e.g., titanium alloy), poly-ether-ether-ketone (PEEK), and the like. In some examples, the burr hole cover 200 may be formed through successive fusion of chosen parts of powder layers applied to a worktable.

In some aspects, the burr hole cover 200 may be fabricated to include a curvature. In other aspects, the burr hole cover 200 may be fabricated without explicitly defining a curvature (whether patient-specific curvature or otherwise). In such other aspects, the burr hole cover 200 may be designed substantially flat (e.g., without a curvature), and the curvature may automatically be induced in the burr hole cover 200 by the pliable/flexible nature of the material used to fabricate the burr hole cover 200. For example, a burr hole cover 200 may be substantially flat and constructed of titanium. Upon affixing this flat, titanium burr hole cover 200 (after drilling surgical fixation devices into the apertures 205) within the recessed region 106, the fixation may automatically induce a curvature of the burr hole cover 200 due to the flexible nature of titanium. In other scenarios, the surgeon, before affixing the substantially flat burr hole cover 200, may apply some force (e.g., by hand) to induce/impart a curvature of the burr hole cover 200.

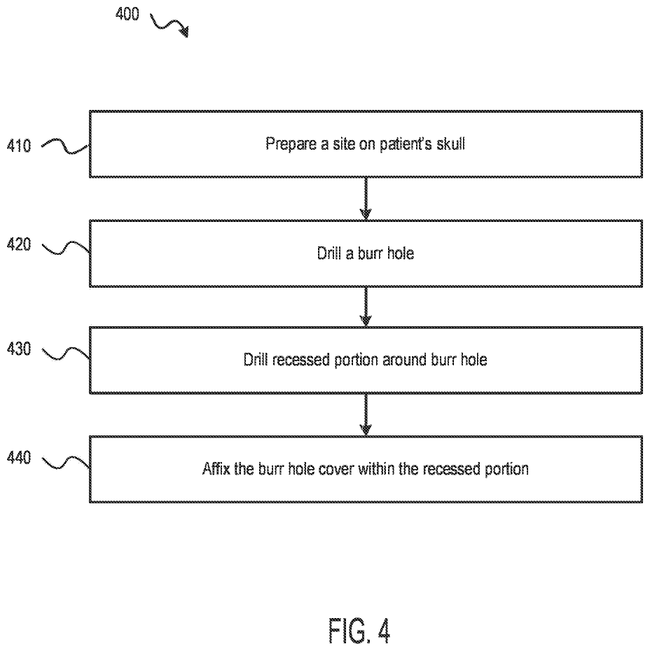

FIG. 4, illustrates a flowchart of an example method 400 for affixing a burr hole cover (e.g., the burr hole cover 200) during a craniotomy procedure. In at least one example, method 400 may be performed by a surgeon or other suitable medical professional. Although the example method 400 is described with reference to the flowchart illustrated in FIG. 4, it will be appreciated that many other methods of performing the acts associated with the method 400 may be used. For example, the order of some of the blocks may be changed, certain blocks may be combined with other blocks, and some of the blocks described are optional.

In one example, the method 400 may begin with exposing a desired portion of the skull by preparing the desired portion (e.g., a site) on the patient's skull (block 410). For instance, a surgeon may first incise the scalp above the desired portion of the skull with a scalpel. The surgeon may then expose the desired portion using a retractor and remove the periosteal layer over the desired portion. One or more burr holes (e.g., the burr hole 100) may then be formed (e.g., drilled) in the skull (block 420). The number of burr holes 100 formed may depend on the type of neurosurgical procedure being performed. In this example, the surgeon drills a single burr hole 100. The burr hole 100 may be formed by using a high-speed drill (e.g., craniotome) which drills a small hole (e.g., 7-9 mm in diameter). Following forming the burr hole 100, the surgeon may perform the desired neurosurgical procedure, which may include one of the many intracranial procedures that may be performed by the surgeon.

After performing the procedure, a recessed region (e.g., the recessed region 106) may be formed (e.g., drilled) around the burr hole 100 (block 430). As described above, the recessed region 106 is a counter bored portion of bone about a center point. In various aspects, the recessed region 106 may be formed with the craniotome using a different kind of drill bit as used when forming the burr hole 100, or may be formed with a different suitable drilling tool. He suitable drilling tool may include a large diameter member that is to rotate around a central axis without translation and create a circular recess through a plunge motion, or may be a smaller diameter tool, either round or non-round, that when rotated can be translated across the bone to create a recess of any shape.

A burr hole cover (e.g., the burr hole cover 200) may then be affixed, or secured, within the recessed region 106 (block 440). For instance, the burr hole cover 200 may be placed within the recessed region 106. Holes for surgical screws may then be drilled in the recessed region 106 through apertures (e.g., the apertures 205) defined in the burr hole cover 200. Surgical screws may then be installed through the apertures 205 and into the drilled holes to affects, or secure, the burr hole cover 200.

The example method 400 described above recites drilling a burr hole 100 and performing the desired neurosurgical procedure before forming the recessed region 106. In other examples, after preparing the drilling site (e.g., block 410), the surgeon may form a recessed region 106 within the drilling site and then drill the burr hole 100 through the recessed region 106. In such other examples, the surgeon performs the desired neurosurgical procedure after forming the recessed region 106 and the burr hole 100. In another example still, both the recessed region 106 and the burr hole 100 may be formed simultaneously using specific bits configured to form both the recessed region 106 and the burr hole 100.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

1. A method for affixing a burr hole cover to a skull bone and over a burr hole, the method comprising:
preparing a site on a patient for the burr hole;
forming a burr hole in the skull bone of the patient at the prepared site;
forming a recessed region in the skull bone of the patient at the prepared site;
positioning a burr hole cover within the formed recessed region; and
installing a respective surgical fixation device through each of one or more apertures of the positioned burr hole cover and into the skull bone to thereby affix the burr hole cover to the skull bone,
wherein the recessed region of the skull bone is around the burr hole.

2. The method of claim 1, wherein the recess region is formed prior to forming the burr hole.

3. The method of claim 1, wherein the burr hole is formed prior to forming the recessed region.

4. The method of claim 1, wherein the formed recessed region has a uniform depth.

5. The method of claim 1, wherein the burr hole cover is constructed with a predefined curvature prior to positioning the burr hole cover within the formed recess region.

6. The method of claim 1, wherein the burr hole cover is flat prior to positioning the burr hole cover within the formed recess region, and wherein installing a respective surgical fixation device through each of the one or more apertures of the positioned burr hole cover and into the skull bone thereby induces a curvature of the burr hole cover.

7. The method of claim 1, wherein the recessed region is formed such that when the burr hole cover is affixed to the skull bone within the recessed region, a clearance between an edge of the burr hole cover and a wall of the recessed region is between 0.00 mm and 0.04 mm.

8. The method of claim 1, wherein the formed recessed region has two separate segments.

9. The method of claim 1, wherein the recessed region is formed with a width, the width being substantially perpendicular to a central axis of the burr hole, and wherein the width of the recessed region is uniform around the burr hole.

10. A burr hole cover for affixation within a recessed region formed in a skull bone to thereby cover a burr hole formed in the skull bone, the burr hole cover comprising:
a contact surface configured to contact the skull bone;
a top surface opposite the contact surface; and
one or more apertures extending from the top surface to the contact surface, each of the one or more apertures configured to receive a surgical fixation device such that when the surgical fixation device is fully inserted into a respective aperture, to thereby affix the burr hole cover to the skull bone, the surgical fixation device is at or below the top surface.

11. The burr hole cover of claim 10, wherein when the burr hole cover is affixed within the recessed region, the top surface is substantially flush with a surface of the skull bone.

12. The burr hole cover of claim 10, wherein each of the one or more apertures includes a sidewall that is inwardly beveled.

13. The burr hole cover of claim 10, wherein the contact surface has a contour that matches a contour of the skull bone.

14. The burr hole cover of claim 10, wherein the top surface has a contour that matches a contour of the skull bone.

15. The burr hole cover of claim 10, wherein the burr hole cover is constructed of titanium.

16. The burr hole cover of claim 10, wherein the burr hole cover has a non-uniform thickness, the thickness being measured from the top surface to the contact surface.

17. The burr hole cover of claim 10, wherein the burr hole cover includes one or more perforations separate from the one or more apertures.

18. The burr hole cover of claim 17, wherein at least one perforation is sized such that a medical instrument may be positioned through the at least one perforation.

19. The burr hole cover of claim 17, wherein at least one perforation is sized such that air and/or moisture may flow through the at least one perforation when the burr hole cover is affixed within the recessed region.

20. The burr hole cover of claim 10, wherein the burr hole cover is constructed from additive manufacturing or freeform fabrication.
